(19) United States
(12) Patent Application Publication
(10) Pub. No.: US 2021/0395465 A1
(43) Pub. Date: Dec. 23, 2021

(54) ABSORPTION AND FILTRATION MEDIA

(71) Applicant: Wool Research Organisation of New Zealand Incorporated, Shirley, Christchurch (NZ)

(72) Inventors: Robert James McClelland Kelly, Christchurch (NZ); Amy Clare Cruickshank, Lincoln (NZ); Gail Louise Krsinic, Springston (NZ)

(21) Appl. No.: 17/283,486

(22) PCT Filed: Oct. 18, 2019
(86) PCT No.: PCT/NZ2019/050139

§ 371 Date: Apr. 7, 2021

(30) Foreign Application Priority Data
Oct. 19, 2018 (NZ) 747490

(51) Int. Cl. C08H 1/06; B01D 39/16; B01D 53/04; B01D 46/00; A24D 3/06; A24D 3/08; C02F 1/28; D06M 11/34; D06M 11/50; D21H 13/34; D21C 9/16; D21C 9/10; B01J 20/24; B01J 20/28; B01J 20/30; A61L 15/32; F16L 59/04

(52) U.S. Cl. CPC C08H 1/06; C02F 2101/30; B01D 53/04; B01D 46/0036; A24D 3/066; A24D 3/08; C02F 1/286; D06M 11/34; D06M 11/50; D21H 13/34; D21C 9/163; D21C 9/1073; B01J 20/24; B01J 20/28023; B01J 20/3085; A61L 15/32; F16L 59/04; B01D 2253/202; B01D 2257/302; B01D 2257/404; B01D 2257/7022; B01D 39/1669

(57) ABSTRACT

Disclosed are keratin fibre cellular components, specifically keratin fibre cuticle and cortical cells, and their use as absorption and filtration media, and in thermal insulation materials. The keratin fibre cellular components may be oxidised. The keratin fibre cellular components have improved absorbency and filtration capacity compared to the source keratin fibres. The keratin fibre cellular components may be used in, for example, various products for passive absorption and active filtration of gas or liquid media.

### FIELD OF THE INVENTION

The present invention relates to keratin fibre cellular components, specifically keratin fibre cuticle and cortical cells, and their use as absorption and filtration media, and in thermal insulation materials.

### BACKGROUND

Fibrous proteins (also known as scleroproteins) are generally inert and insoluble in water. Fibrous proteins form long protein filaments shaped like rods or wires. They are structural or storage proteins. Fibrous proteins include keratin, collagen, elastin and fibroin.

Keratin fibres include wool, fur, hair and feathers. Wool is a keratin fibre produced by various animals including sheep, goats, camels and rabbits. The fibre structure comprises a cuticle, cortex, and medulla, although fine wools may lack the medulla.

The diameter of sheep wool typically ranges from about 10 microns to about 45 microns. Fibre diameter is an important characteristic of wool in relation to its quality and price. Finer wools are softer and suitable for use in garment manufacturing. There are a limited number of consumer applications remaining for stronger wool types such as flooring, bedding, upholstery, and hand knitting yarns.

Wool comprises three main histological components; two cellular components and a cell membrane complex that is present between the cells and maintains the structure together. The cellular components are cortical cells, which comprise the internal structure of the fibre, and cuticle cells, which overlap to form the outer layer. This complex biological assembly is created during wool growth by the body in the follicle.

A variety of methods are known for degrading wool fibres to release cellular components as cortical cells. A wide range of potential uses for isolated cortical cells have been suggested, including feedstuffs, fertilizer and hair care products, and in bio-composite materials.

Wool-based materials such as loose fibres, fabrics, keratin powders or colloidal solutions and composite wool keratin-polymer nanofiber membranes can be useful absorbent materials for removing volatile pollutant compounds (for example, formaldehyde, sulfur dioxide and nitrogen dioxide) from the atmosphere and heavy-metal ions or organic compounds from solution.

But, while the absorption and filtration properties of wool are known, the use of wool fibres in such applications is limited by the physical form and dimensions of the wool fibre restricting modes of use, and the limited capacity of wool to absorb and filter liquid and gaseous pollutants.

Accordingly, it is an object of the present invention to go some way to avoiding the above disadvantages; and/or to at least provide the public with a useful choice.

Other objects of the invention may become apparent from the following description which is given by way of example only.

### SUMMARY OF THE INVENTION

In a first aspect, the present invention provides a process for oxidising keratin fibre cellular components, the process comprising:

(a) providing keratin fibre cellular components; and
(b) contacting the keratin fibre cellular components with an oxidant to provide oxidised keratin fibre cellular components.

In one embodiment, the keratin fibre cellular components are a combination of keratin fibre cuticle and cortical cells. In one embodiment, the keratin fibres are selected from wool, fur and hair. In one embodiment, the keratin fibres are wool. In a preferred embodiment, the wool is sheep wool.

In one embodiment, the oxidant is selected from hydrogen peroxide and ozone. Ozone is preferred.

The keratin fibre cellular components may be substantially dry prior to being contacted with ozone. Alternatively, the keratin fibre cellular components may be wet prior to being contacted with ozone. Accordingly, the process may further comprise:

(c) drying the oxidised keratin fibre cellular components.

The invention also provides oxidised keratin fibre cellular components when produced by a process of the invention. The invention also provides oxidised keratin fibre cellular components obtainable by a process of the invention.

In a second aspect, the present invention provides oxidised keratin fibre cellular components.

In a third aspect, the present invention provides an absorbent product comprising keratin fibre cellular components. In one embodiment, the keratin fibre cellular components comprise oxidised keratin fibre cellular components.

In one embodiment, the product is a liquid absorbent product. The product may be used for absorbing blood and/or urine. In one embodiment, the product is a personal hygiene product. In another embodiment, the product is a medical product.

In one embodiment, the product is a gas absorbent product. The product may be a composite foam. Alternatively, the product may be a network structure or a paper. In one embodiment, the product is for passive absorption of gaseous pollutants. In one embodiment, the gas is selected from SO₂, NO₂, CH₂O, or a mixture of any two or more thereof.

Another aspect of the present invention provides a filter comprising keratin fibre cellular components. In one embodiment, the keratin fibre cellular components comprise oxidised keratin fibre cellular components. The keratin fibre cellular components may be comprised in a composite foam, a network structure or a paper. The filter may be a liquid filter or a gas filter. In one embodiment, the filter is a cigarette filter.

Another aspect of the present invention provides a method of decreasing the concentration of a pollutant in a gas stream, the method comprising passing the gas stream through a filter comprising keratin fibre cellular components. In one embodiment, the keratin fibre cellular components comprise oxidised keratin fibre cellular components. In one embodiment, the pollutant is selected from SO₂, NO₂, CH₂O, or a mixture of any two or more thereof.

Another aspect of the present invention provides a method of decreasing the concentration of a pollutant in a liquid stream, the method comprising passing the liquid stream through a filter comprising keratin fibre cellular components. In one embodiment, the keratin fibre cellular components comprise oxidised keratin fibre cellular components. The pollutant may be a metal ion.

Another aspect of the present invention provides a method for absorbing a pollutant from a gas, the method comprising contacting the gas with a material comprising keratin fibre cellular components. The present invention also provides a method for decreasing the concentration of a pollutant in a gas, the method comprising contacting the gas with a material comprising keratin fibre cellular components. In one embodiment, the keratin fibre cellular components comprise oxidised keratin fibre cellular components. The material may be a composite foam. Alternatively, the material may be a network structure or a paper. In one embodiment, the pollutant is selected from SO₂, NO₂, CH₂O, or a mixture of any two or more thereof.

Another aspect of the present invention provides a method for absorbing a pollutant from a liquid, the method comprising contacting the liquid with a material comprising keratin fibre cellular components. The present invention also provides a method for decreasing the concentration of a pollutant in a liquid, the method comprising contacting the liquid with a material comprising keratin fibre cellular components. In one embodiment, the keratin fibre cellular components comprise oxidised keratin fibre cellular components. In one embodiment, the pollutant is a metal ion.

Another aspect of the present invention provides a method for absorbing a metal ion from a liquid, the method comprising contacting the liquid with a material comprising keratin fibre cellular components. The present invention also provides a method for decreasing the concentration of a metal ion in a liquid, the method comprising contacting the liquid with a material comprising keratin fibre cellular components. In one embodiment, the keratin fibre cellular components comprise oxidised keratin fibre cellular components. The keratin fibre cellular components may be comprised in a composite foam, a network structure or a paper.

Another aspect of the present invention provides use of keratin fibre cellular components for decreasing the concentration of a pollutant in a gas. The present invention also provides use of keratin fibre cellular components for absorbing a pollutant from a gas. In one embodiment, the keratin fibre cellular components comprise oxidised keratin fibre cellular components. The keratin fibre cellular components may be comprised in a composite foam. Alternatively, the keratin fibre cellular components may be comprised in a network structure or a paper. In one embodiment, the pollutant is selected from SO₂, NO₂, CH₂O, or a mixture of any two or more thereof.

Another aspect of the present invention provides use of keratin fibre cellular components for decreasing the concentration of a pollutant in a liquid. The present invention also provides use of keratin fibre cellular components for absorbing a pollutant from a liquid. In one embodiment, the keratin fibre cellular components comprise oxidised keratin fibre cellular components. The keratin fibre cellular components may be comprised in a composite foam, a network structure or a paper. The pollutant may be a metal ion. The keratin fibre cellular components may be comprised in a composite foam, a network structure or a paper.

Another aspect of the present invention provides use of keratin fibre cellular components for decreasing the concentration of a metal ion in a liquid. The present invention also provides use of keratin fibre cellular components for absorbing a metal ion from a liquid. In one embodiment, the keratin fibre cellular components comprise oxidised keratin fibre cellular components. The keratin fibre cellular components may be comprised in a composite foam, a network structure or a paper.

Another aspect of the present invention provides a network structure comprising keratin fibre cellular components. In one embodiment, the keratin fibre cellular components comprise oxidised keratin fibre cellular components. The keratin fibre cellular components may be bound with an adhesive.

Another aspect of the present invention provides a thermal insulation material comprising keratin fibre cellular components. The present invention also provides use of keratin fibre cellular components as a thermal insulation material. In one embodiment, the keratin fibre cellular components comprise oxidised keratin fibre cellular components. The keratin fibre cellular components may be comprised in a network structure.

Another aspect of the present invention provides a paper comprising keratin fibre cellular components. In one embodiment, the keratin fibre cellular components comprise oxidised keratin fibre cellular components.

Although the present invention is broadly as defined above, those persons skilled in the art will appreciate that the invention is not limited thereto and that the invention also includes embodiments of which the following description gives examples.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### DETAILED DESCRIPTION

As used herein “(s)” following a noun means the plural and/or singular forms of the noun.

As used herein the term “and/or” means “and” or “or” or both.

The term “comprising” as used in this specification means “consisting at least in part of”. When interpreting statements in this specification which include that term, the features, prefaced by that term in each statement or claim, all need to be present but other features can also be present. Related terms such as “comprise”, “comprises” and “comprised” are to be interpreted in the same manner.

The present invention broadly relates to the use of keratin fibre cellular components as absorption and filtration media. The present invention also relates to the use of keratin fibre cellular components in thermal insulation materials.

The term “keratin fibre cellular components” as used in this specification means keratin fibre cuticle cells, keratin fibre cortical cells, or a combination of keratin fibre cuticle and cortical cells. Preferably, the keratin fibre cellular components are a combination of keratin fibre cuticle and cortical cells.

The present description is substantially directed to keratin fibre cellular components obtained from wool. However, the invention is not limited thereto and cellular components obtained from other keratin fibres, such as hair, fur and feathers, are also useful in the present invention. In a preferred embodiment, the keratin fibres are wool, hair, or fur, or a mixture of any two or more thereof. In a preferred embodiment, the wool is sheep wool.

The keratin fibre cellular components of the present invention have been found to be effective at absorbing and filtering a range of gas and liquid pollutants, and so are suitable for use in, for example, various products for passive absorption and active filtration of gas or liquid media. The keratin fibre cellular components of the present invention have a high surface area and provide a highly functional material. Advantageously, the keratin fibre cellular components of the present invention can be formed into products that are not limited by the physical form and/or dimensions of the source keratin fibres. In addition, the keratin fibre cellular components have improved absorbency and filtration capacity compared to the source keratin fibres.

The keratin fibre cellular components of the present invention may be prepared by methods known to those persons skilled in the art.

In one embodiment, keratin fibre cellular components are prepared from keratin fibres using a combination of enzymatic action followed by mechanical disruption, preferably by mixing at high shear rates. The combination disrupts the keratin fibre structure and converts keratin fibres into a loose combination of cuticle and cortical cells.

A range of proteolytic enzymes may be used to prepare keratin fibre cellular components from keratin fibres, including papain, trypsin and the protease from Bacillus licheniformis. In one embodiment, the protease from Bacillus licheniformis is used.

Scanning electron microscopy analysis of the keratin fibre cellular components obtained from wool using the protease from Bacillus licheniformis, for example, showed that the cellular components contain no intact wool fibres, but instead are a loose collection of cuticle and cortical cells. That is, the enzyme assisted in achieving complete conversion of the wool fibres into wool cellular components. The wool cellular components comprise a significantly higher proportion of cortical cells than cuticle cells because of the naturally higher abundance of cortical cells in the wool fibre.

Typical dimensions of the wool cortical cells were determined using microscopy. The wool cortical cells have an ellipsoid shape and are typically 70-120 microns long with a diameter of 4-8 microns.

Without wishing to be bound by theory, it is thought the primary action of the enzyme is to disrupt the cell membrane complex within the keratin fibres leading to a weakening of the structure. Accordingly, the process typically requires maintaining the keratin fibres under pH and temperature conditions suitable for enzyme activity. In one embodiment, the temperature is about 25° C. to about 70° C. Preferably, the temperature is about 65° C. In one embodiment, the pH is about 7.5 to about 8.5. Preferably, the pH is about 8.5.

The enzyme may be added to the keratin fibres in one or more aliquots.

The keratin fibres are contacted with the enzyme for a time sufficient to weaken the keratin fibres so that the keratin fibres are susceptible to mechanical disruption. In one embodiment, the time is about 20 hours to about 36 hours. Preferably, the time is about 24 hours.

Following the enzymatic action, the keratin fibres are then disassembled into their cellular components by mechanical disruption, preferably by high shear mixing. The invention is not, however, limited thereto and other forms of mechanical disruption such as ultrasound and reflux disruption may be used, either alone or in any combination.

In another embodiment, keratin fibre cellular components are prepared from keratin fibres using a combination of chemical action followed by mechanical disruption, preferably by mixing at high shear rates. Again, the combination disrupts the keratin fibre structure and converts keratin fibres into a loose combination of cuticle and cortical cells.

Chemical agents suitable for use in this embodiment swell the keratin fibre and include, but are not limited to, formic acid, dimethyl sulfoxide and urea. Formic acid is preferred.

Without wishing to be bound by theory, it is thought these chemical agents disrupt the keratin fibre structure, swelling the fibre and penetrating into the cell membrane complex.

In one embodiment, the chemical agent is formic agent. A relatively high concentration of formic acid is preferred, typically at least about 80% and preferably about 98%.

The keratin fibres are contacted with the chemical agent for a time sufficient to weaken the keratin fibres so that the keratin fibres are susceptible to mechanical disruption. Those persons skilled in the art will appreciate that the time can vary with different chemical agents. In one embodiment, the time is about 30 minutes to about three hours. Preferably, the time is about one hour. In one embodiment, the keratin fibres are contacted with the chemical agent at a temperature of about 20° C. to about 40° C. Preferably, the keratin fibres are contacted with the chemical agent at a temperature of about 20° C.

Following the chemical action, the keratin fibres are then disassembled into their cellular components by mechanical disruption, preferably by high shear mixing. The invention is not, however, limited thereto and other forms of mechanical disruption such as ultrasound and reflux disruption may be used, either alone or in any combination.

In an alternative embodiment of the above enzymatic and chemical processes, ultrasound is used instead of or in addition to high shear mixing to provide the mechanical disruption required to deconstruct the keratin fibres into their cellular components. In a further alternative embodiment, reflux disruption is used instead of or in addition to high shear mixing to provide the mechanical disruption required to deconstruct the keratin fibres into their cellular components.

In one embodiment of the above enzymatic and chemical processes, wherein the keratin fibre cellular components consist essentially of cuticle cells, the mechanical disruption is selected from ultrasound and reflux disruption.

In one embodiment of the above enzymatic and chemical processes, the keratin fibres are pre-treated before the enzymatic or chemical action. The pre-treatment may remove, or at least partially remove, the cuticle from the keratin fibres, or otherwise disrupt the surface of the keratin fibres.

In one embodiment, the pre-treatment comprises ultrasound, milling and/or abrasive removal. Suitable abrasives include, but are not limited to, carbon powder, glass fibres, and glass beads. Abrasive removal may include the use of stirrers and/or vortex equipment.

In another embodiment, the pre-treatment comprises oxidation. Suitable oxidants include hydrogen peroxide and ozone.

The keratin fibre cellular components may be isolated from the mixture obtained following mechanical disruption by methods known to those persons skilled in the art. In one embodiment, the liquid mixture obtained following mechanical disruption is filtered to isolate the keratin fibre cellular components. For example, a 63 micron mesh sieve may be used to isolate the keratin fibre cellular components. In another embodiment, the keratin fibre cellular components are isolated by centrifuging the mixture obtained following mechanical disruption.

The isolated keratin fibre cellular components may be dried by any suitable method. In one embodiment, the keratin fibre cellular components are dried at elevated temperature in an oven. In one embodiment, the keratin fibre cellular components are dried at a maximum temperature of about 100° C. Preferably, the keratin fibre cellular components are dried at a temperature of about 65° C. to about 85° C. In an alternative embodiment, the keratin fibre cellular components are dried by lyophilisation.

Typically, drying produces a dried mass of keratin fibre cellular components. The dried mass may conveniently be comminuted using, for example, an agitator or blender, such as a blade in a food processor. The process is not limited thereto, and other dry milling techniques known to those skilled in the art may also be used. Dry sieving may also be used to fractionate the resulting powder into different particle size fractions.

The resulting keratin fibre cellular components are suitable for use in a variety of applications according to the invention.

In particular, the keratin fibre cellular components have properties that are advantageous for use in the absorption and filtration of a range of gases and liquids.

The keratin fibre cellular components are also useful in thermal insulation materials.

The keratin fibre cellular components are light weight with a low bulk density. For example, wool cellular components have a bulk density of about 33 cm³ per gram; similar to that of the source wool from which they were obtained. However, the surface area of the wool cellular components is significantly greater (about 900 times) than that of wool.

This increased surface area greatly enhances those characteristics related to the surface properties of the keratin fibre cellular components compared to the keratin fibres. Moreover, it has surprisingly been found that the surface characteristics of the keratin fibre cellular components are different to those of the keratin fibres. For example, a drop of water placed on a wool surface was observed to bead for more than 300 seconds prior to spreading as it is absorbed by the wool fibres. A drop of water placed on wool cellular components did not bead and, instead, spread instantly. Without wishing to be bound by theory, this difference is thought to be the result of the different surface characteristics of the wool cellular components compared to wool. The removal of the outer lipid layers and the cell membrane complex from the wool fibre to provide the wool cellular components is thought to expose the wool proteins to the surface, such that the wool cellular components have a much more hydrophilic surface than wool.

Accordingly, keratin fibre cellular components are useful in domestic, commercial and industrial products requiring a material with liquid absorbent properties. More particularly, keratin fibre cellular components may be used in combination with, or instead of, conventional absorbent materials currently used in these products; such as sodium polyacrylate polymers and starch based absorbents.

Such products may be useful for absorbing biological fluids, including but not limited to urine and blood.

In one embodiment, the product is a personal hygiene product. Such personal hygiene products include, but are not limited to, infant or adult diapers and incontinence products and liners, tampons and feminine care absorbent pads.

In another embodiment, the product is for absorbing blood. Such products may include various of the personal hygiene products noted above, as well as medical products, such as medical sponges, wound dressings and surgical dressings, including haemostatic dressings, used for blood absorption, for example during surgery or after trauma.

In one embodiment, the keratin fibre cellular components are contacted with an oxidant. Suitable oxidants include hydrogen peroxide and ozone. Ozone is preferred. In one embodiment, the concentration of ozone is about 160 ppm to about 180 ppm. In one embodiment, the ozone is mixed with air. In one embodiment, the keratin fibre cellular components are contacted with ozone for about 60 minutes to about 180 minutes. In another embodiment, the keratin fibre cellular components are contacted with ozone for about 180 minutes.

The keratin fibre cellular components may be contacted with ozone after isolation and while wet, or after drying. Generally, when wet, the keratin fibre cellular components typically comprise about 80% (w/w) moisture. After drying, the keratin fibre cellular components comprise about 15% (w/w) moisture. The invention is not, however, limited to these moisture contents and keratin fibre cellular components with other moisture contents may also be used.

Oxidation of wool cellular components has been found to significantly increase their ability to absorb water or biological fluids, such as blood or saline. Without wishing to be bound by theory, this increase in liquid absorbency is thought to be due to the oxidation of amino acid groups within and on the surface of the keratin fibre cellular components providing a more polar material. For example, the amino acid cystine may be oxidized to cysteic acid by an oxidant, increasing the polarity and, therefore, the liquid absorbency of the keratin fibre cellular components.

Surprisingly, following oxidation with ozone, the liquid absorbency of wool cellular components has been found to increase by about 30% in saline absorption under load (AUL) testing. In contrast, wool showed no increase in saline AUL testing following oxidation with ozone.

In addition to their increased liquid absorbency compared to intact keratin fibres, keratin fibre cellular components have surprisingly been found to have significantly increased gas absorbency compared to intact keratin fibres. For example, wool cellular components have been found to be much more effective materials for the passive absorption and removal of gaseous pollutants (such as sulfur dioxide, nitrogen dioxide and formaldehyde) compared to intact wool.

Keratin fibre cellular components may be used for the passive absorption of pollutants by incorporating the keratin fibre cellular components into various materials that form part of an environment. Such materials including the keratin fibre cellular components could form part of an indoor or outdoor environment, thereby improving the air quality of that environment.

In this and other embodiments, the keratin fibre cellular components may form, for example, a sheet, a membrane or a material. Alternatively, the keratin fibre cellular components may be incorporated in a sheet, in a membrane or in a material, such as a foam or composite.

For example, keratin fibre cellular components may be included in a composite foam. In one embodiment, keratin fibre cellular components are included in a flexible polyurethane foam, the keratin fibre cellular components comprising about 5% of the foam by mass. Such a foam comprising wool cellular components was found to absorb 5% more nitrogen dioxide gas that an identical foam containing no wool cellular components.

Alternatively, keratin fibre cellular components may be included in a paper. Advantageously, keratin fibre cellular components may be used as a substitute for a portion of the cellulose pulp (e.g. wood pulp) in a conventional paper making process. In one embodiment, the paper comprises about 1% to about 80% or about 10% to about 80%, or about 20% to about 80%, or about 30% to about 80%, or about 40% to about 80%, or about 50% to about 80%, or about 60% to about 80% by mass of the keratin fibre cellular components. In a preferred embodiment, the paper comprises about 70% by mass of the keratin fibre cellular components.

As a further alternative, keratin fibre cellular components may be included in a network structure, in which the keratin fibre cellular components are bound together by an adhesive. In one embodiment, the network structure comprises about 50% to about 90%, or about 60% to about 90%, or about 65% to about 90%, or about 66% to about 89%, or about 70% to about 90% by mass of the keratin fibre cellular components. In one embodiment, the network structure comprises about 80% by mass of the keratin fibre cellular components.

Suitable adhesives for use as the binder in the network structure will be apparent to those persons skilled in the art, and include, but are not limited to, epoxies, cyanoacrylates, poly vinyl acetates, ethylene vinyl acetates, polyurethanes, soluble proteins, poly lactic acids, including low melt temperature poly lactic acid, low melt temperature polyesters, starches, celluloses and other spray adhesives. In a preferred embodiment, the adhesive is a cyanoacrylate.

In addition to their utility in the passive absorption and removal of gaseous pollutants, keratin fibre cellular components have surprisingly been found to be useful for the active filtration and removal of gaseous pollutants. For example, wool cellular components have been found to be much more effective materials for the active filtration and removal of gaseous pollutants (such as sulfur dioxide, nitrogen dioxide and formaldehyde) compared to intact wool. Wool cellular components have also been found to be effective materials for the active filtration and removal of vapours, such as oil vapour.

Accordingly, keratin fibre cellular components may be used for active filtration of pollutant gases by incorporating the keratin fibre cellular components into gas filtration devices, either alone or in combination with other filter media. Similarly, the keratin fibre cellular components may be used for active filtration of vapours such as pollutant vapours by incorporating the keratin fibre cellular components into gas filtration devices, either alone or in combination with other filter media.

In one embodiment, the keratin fibre cellular components are incorporated in personal protection equipment, such as work place gas masks, personal filtration face masks for use outdoors to protect against urban pollution, or in other filtration apparatus for the flow of gas to the mouth and/or nose to facilitate breathing. In another embodiment, the keratin fibre cellular components are incorporated in filtration apparatus for indoor air, such as filtration apparatus used in home and/or industrial air ventilation for reduction of noxious gases, vapours, particles and odours.

In another embodiment, the keratin fibre cellular components are used for vapour and/or odour control in domestic or commercial cooking environments. For example, the keratin fibre cellular components may be used for moisture and/or oil vapour removal in range hoods or other forced gas filtration systems. The invention is not, however, limited thereto and the keratin fibre cellular components may be used for moisture and/or oil vapour removal in other domestic, commercial or industrial applications.

In view of their utility in absorbing and removing pollutants such as formaldehyde, the keratin fibre cellular components may also be used to replace standard cellulose filters in cigarettes. Such filters advantageously also capture particulates and tar from smoke drawn through the filter during use.

Keratin fibre cellular components have also surprisingly been found to be effective at removing pollutants, particularly metal ions, from aqueous systems. In one embodiment, the metal ions are copper ions.

In another embodiment, the keratin fibre cellular components are used for thermal insulation. For example, the network structure described above may be used instead of, or in addition to, conventional insulation such as polyester or down. Advantageously, the network structure comprising the keratin fibre cellular components may be formed into material suitable for use as, for example, padding, batting or wadding.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

The following non-limiting examples are provided to illustrate the present invention and in no way limit the scope thereof.

### EXAMPLES

### Example 1—Preparation of Wool Cellular Components

### Example 1a—Enzymatic

A batch of wool cellular components was prepared from 450 g of wool using the following procedure:
in a 12 L vessel, premix a 10 L solution of 1.5 g/L sodium metabisulfite and 0.5 g/L citric acid and heat to 65° C.;
adjust the premix pH to 8.5 with dilute sodium hydroxide;
add 5% on mass of Protex 6L (a bacterial alkaline protease derived from a selected strain of Bacillus licheniformis);
add 450 g of clean chopped wool to the solution and immerse for 8 hours at pH 8.5 and 65° C.;
add 1% on mass of Protex 6L to the vessel and leave fully immersed for a further 16 hours at pH 8.5 and 65° C.;
mix the slurry in the vessel using high shear for 30 minutes with 55 mm diameter mixing head at about 13000 rpm using an open tooth rotor appropriate for fibrous material;
transfer the mixture to a mesh filter and sieve through a 63 micron screen;
rinse with water;
freeze dry the retentate;
loosen the resulting sheet of dried wool cellular components in a food processor.

### Example 1b—Formic Acid

0.8 g wool with a snippet length of about 10 mm was immersed in 80 ml of 98% formic acid in a large boiling tube at room temperature and left for 1 hour.

The resulting mixture was processed using a high shear dispersing probe at 18000 rpm, with an open tooth rotor for fibrous material, with high shear mixing for 20 cycles of 1 minute bursts with 1 minute cooling in an ice bath between bursts.

The resulting slurry was then sieved through 63, 32 and 20 micron mesh sieves. The majority of the cortical cells were collected in the 63 micron sieve.

The subsequent examples used wool cellular components prepared by the method of Example 1a.

### Example 2—Absorption/Filtration of Sulfur Dioxide (SO₂) Gas

### Example 2a—Absorption of SO₂ by Wool Cellular Components

The absorption of SO₂ by wool cellular components was measured using a glass chamber (3.5 L volume) containing 5 g of wool cellular components inside a metal wire mesh cage and a portable air quality monitoring device with an interchangeable SO₂ sensor. The SO₂ gas absorption capacity of intact wool fibres and the control system (without wool cellular components or wool fibres in the metal wire mesh cage) were also tested for comparison.

SO₂ flowed into the chamber (with or without wool cellular components or wool fibres) from a commercially supplied gas cylinder (10 ppm SO₂) for 4.5 minutes. The chamber was then closed and the decrease in gas concentration inside the sealed chamber was subsequently monitored. In the absence of wool cellular components or wool fibres, the concentration of SO₂ detected in the chamber at 4.5 minutes was about 10 ppm (the nominal maximum detection limit of the SO₂ sensor). After the chamber was closed, a small increase in SO₂ concentration was detected (maximum concentration 11.5 ppm at 5.5 minutes) before the gas concentration decayed to 5.2 ppm at 25 minutes, demonstrating that the control system (without wool cellular components or wool fibres) absorbed some SO₂.

In the presence of intact wool fibres, the absorption of SO₂ gas was more rapid. The maximum SO₂ concentration measured before the chamber was closed was lower with wool fibres in the chamber (5.7-5.8 ppm at 4.5 minutes). This is attributed to the wool fibres absorbing SO₂ gas as it entered the chamber. After the chamber was closed, a small increase in SO₂ concentration was detected (6.2-6.5 ppm at 5-5.5 minutes) before the gas concentration decayed to 0.2-0.3 ppm at 25 minutes.

The wool cellular components absorbed more SO₂ than intact wool fibres. The maximum SO₂ concentration measured before the chamber was closed was lower with wool cellular components in the system (4.5-5.4 ppm at 4.5 minutes). This is attributed to the wool cellular components absorbing more SO₂ gas as it entered the chamber. After the chamber was closed, a small increase in SO₂ concentration was detected (4.9-5.7 ppm at 5 minutes) before the gas concentration decayed to 0.05 ppm at 25 minutes.

In summary, 99% of SO₂ gas was absorbed by wool cellular components in the sealed chamber over 25 minutes, compared to 55% of the gas when no wool cellular components nor wool were present and 95% of the gas when the same mass of intact wool was present.

### Example 2b—Filtration of SO₂ by Wool Cellular Components

The filtration of SO₂ by wool cellular components was measured using a ‘filtration tube’ consisting of a glass tube with a porous glass frit in the middle. A sample (1 g) was placed inside the glass tube and SO₂ gas (25 ppm) flowed from a commercially supplied cylinder through the glass filtration tube, past the sample, to a chamber containing an Aeroqual air quality monitoring device. The device had an interchangeable SO₂ sensor, which detected the SO₂ gas concentration exiting the filtration set-up.

When wool cellular components (1 g) were present in the filtration tube, a negligible amount of SO₂ gas was detected at the system exit for 30 minutes. This is attributed to the wool cellular components absorbing the majority of the SO₂ gas passing through the system. After 30 minutes, the SO₂ gas concentration detected at the system exit increased steadily until a level of 10 ppm SO₂ was reached at 56 minutes (10 ppm is the maximum detection limit of the SO₂ sensor specified by Aeroqual).

When there was no sample present in the glass filtration tube, the Aeroqual sensor detected 10 ppm SO₂ after about 4 minutes. Furthermore, intact wool fibres were observed to be very poor at filtering SO₂. When wool fibres (1 g) were present in the filtration tube, 10 ppm SO₂ was detected at the system exit after 7 minutes.

### Example 3—Absorption/Filtration of Nitrogen Dioxide (NO₂) Gas

### Example 3a—Absorption of NO₂ by Wool Cellular Components

The absorption of NO₂ by wool cellular components was measured using a glass chamber (3.5 L volume) containing 5 g of wool cellular components inside a metal wire mesh cage and a portable air quality monitoring device with an interchangeable NO₂ sensor. The NO₂ gas absorption capacity of intact wool fibres and the control system (without wool cellular components or wool fibres in the metal wire mesh cage) were also tested for comparison.

NO₂ flowed into the chamber (with or without wool cellular components or wool fibres) from a commercially supplied gas cylinder (5 ppm NO₂) for 2.5 minutes. The chamber was then closed and the decrease in gas concentration inside the sealed chamber was subsequently monitored. In the absence of wool cellular components or wool fibres, the concentration of NO₂ detected in the chamber at 2.5 minutes was 1.21-1.34 ppm (nominal maximum detection limit of the NO₂ sensor was 1 ppm NO₂). After the chamber was closed, a small increase in NO₂ concentration was detected (maximum concentration 1.37-1.42 ppm at 3.5 minutes) before the gas concentration decayed to 0.50-0.62 ppm at 25 minutes, demonstrating that the control system (without wool cellular components or wool fibres) absorbed some NO₂.

The absorption of NO₂ by intact wool fibres was similar to that observed for the control system. With wool fibres in the chamber, the NO₂ concentration detected at 2.5 minutes (before the chamber was closed) was 1.11-1.23 ppm. After the chamber was closed, the NO₂ concentration increased to 1.32-1.33 ppm before decaying to 0.30-0.50 ppm at 25 minutes. Accordingly, the intact wool fibres absorbed only slightly more NO₂ than the control system.

In the presence of wool cellular components, the absorption of NO₂ was considerably more rapid. The maximum NO₂ concentration measured before the chamber was closed was lower with wool cellular components in the chamber (0.59-0.62 ppm at 2.5 minutes). This is attributed to the wool cellular components absorbing NO₂ gas as it entered the chamber. After the chamber was closed, a small increase in NO₂ concentration was detected (0.76 ppm at 3 minutes) before the concentration decayed to 0 ppm at 14-15 minutes.

In summary, 100% of the NO₂ gas was absorbed from the sealed chamber containing wool cellular components over 14-15 minutes compared to 56% of the gas in 25 minutes when no wool cellular components nor wool were present and 59% of the gas when the same mass of intact wool was present.

### Example 3b—Filtration of NO₂ by Wool Cellular Components

The filtration of NO₂ by wool cellular components was measured using a ‘filtration tube’ consisting of a glass tube with a porous glass frit in the middle. A sample (1 g) was placed inside the glass tube and NO₂ gas (5 ppm) flowed from a commercially supplied cylinder through the glass filtration tube, past the sample, to a chamber containing an Aeroqual air quality monitoring device. The device had an interchangeable NO₂ sensor, which detected the NO₂ gas concentration exiting the filtration set-up.

When wool cellular components (1 g) were present in the filtration tube the NO₂ concentration detected at the filtration system exit increased steadily from the beginning of the experiment until the maximum NO₂ sensor detection limit of 1 ppm was reached at 14 minutes.

When there was no sample present in the glass filtration tube, the Aeroqual sensor detected 1 ppm NO₂ after about 5 minutes. Furthermore, intact wool fibres were observed to be very poor at filtering NO₂. When wool fibres (1 g) were present in the filtration tube, 1 ppm NO₂ was detected at the system exit after only 6 minutes.

### Example 4—Absorption/Filtration of Formaldehyde (CH₂O) Gas

### Example 4a—Absorption of Formaldehyde by Wool Cellular Components

The absorption of CH₂O by wool cellular components was measured inside a glass chamber (3.5 L volume) containing 5 g of wool cellular components inside a metal wire mesh cage and a portable air quality monitoring device with an interchangeable CH₂O sensor. The CH₂O gas was generated in situ in a second sealed chamber by heating a 1.25% (w/w) solution of paraformaldehyde dissolved in phosphate buffer solution (pH 7.3) to 30° C. with a hot plate. A vacuum pump was used to pull the CH₂O gas generated into the experimental chamber. The CH₂O gas absorption capacity of intact wool fibres and the control system (without wool cellular components or wool fibres in the metal wire cage) was also examined for comparison.

CH₂O was pulled into the experimental chamber (with or without wool cellular components or wool fibres) for 4 minutes before the chamber was closed and the decrease in CH₂O gas concentration inside the sealed chamber was subsequently monitored. In the absence of wool cellular components or wool fibres, the concentration of CH₂O detected in the chamber at 4 minutes was 8.5-8.8 ppm (the maximum detection limit of the CH₂O sensor was 10 ppm). After the chamber was closed, a small increase in CH₂O concentration was detected (maximum concentration 9.0-9.2 ppm at 5 minutes) before the gas concentration decayed to 6.1-6.4 ppm at 25 minutes, demonstrating that the control system (without wool cellular components or wool fibres) absorbed some CH₂O.

More CH₂O was absorbed when intact wool fibres were present in the chamber. Initially, the maximum CH₂O concentration detected before the chamber was closed (8.0-9.4 ppm at 4 minutes) was similar to that detected for the control system. After the chamber was closed, a further small increase in CH₂O concentration was detected (8.2-10.1 ppm at 5 minutes). The CH₂O concentration then decayed to 1.7-2.0 ppm at 25 minutes, demonstrating that the wool fibres absorbed more CH₂O than the control system.

The absorption of CH₂O gas was considerably more rapid in the presence of wool cellular components. The maximum CH₂O concentration measured before the chamber was closed was much lower with wool cellular components in the chamber (4.0-4.2 ppm at 4 minutes). This is attributed to the wool cellular components absorbing CH₂O gas as it entered the chamber. After the chamber was closed, a small increase in CH₂O concentration was detected (4.5 ppm at 4.5-5 minutes) before the gas concentration decayed to 0.1 ppm at 25 minutes.

In summary, wool cellular components are effective at absorbing formaldehyde; removing 98% of formaldehyde from a sealed chamber in 25 minutes, compared to 41% of the gas in 25 minutes when no wool cellular components nor wool were present and 80% of the gas when the same mass of intact wool was present.

### Example 4b—Filtration of Formaldehyde by Wool Cellular Components

The filtration of CH₂O by wool cellular components was measured using a ‘filtration tube’ consisting of a glass tube with a porous glass frit in the middle. CH₂O gas was generated in situ in a sealed chamber by heating a 4% solution of formaldehyde in phosphate buffer (pH 7.2) to 30° C. with a hot plate. A vacuum pump was used to pull the gas through the filtration tube, past the sample (1 g), and into a second chamber containing an air quality monitoring device with an interchangeable CH₂O sensor which detected the CH₂O gas concentration exiting the filtration set-up.

When there was no sample present in the glass filtration tube, the sensor detected 10 ppm CH₂O after about 1.5-1.75 minutes (the maximum detection limit of the CH₂O sensor was 10 ppm).

Three repeat experiments were performed to measure CH₂O filtration in the presence of wool cellular components (1 g). The CH₂O concentration detected at the filtration system exit during the experiments in the presence of wool cellular components increased steadily from the beginning of the measurement but the total time before the maximum CH₂O sensor detection limit of 10 ppm was detected at the system exit varied widely (26-62 minutes).

This large variation was attributed to changes in room temperature between experiments affecting formaldehyde gas concentration. The amount of formaldehyde gas generated is temperature dependent. While the temperature of the formaldehyde solution to generate the gas was controlled to 30±3° C. during the measurement, the room temperature was not controlled. It is thought the variation in the temperature of the air mixed with the formaldehyde gas when it was pulled through the filtration set-up using the vacuum pump most likely induced changes in formaldehyde gas concentration, which led to the observed variation in the time required to detect 10 ppm formaldehyde at the system exit.

### Example 5—Filtration of Cigarette Smoke

Cigarette filters containing wool cellular components were fabricated by packing loose wool cellular components (0.1 g, the weight of a standard cellulose filter) into the same volume occupied by a cellulose filter removed from a cigarette. Cigarettes containing standard cellulose filters or wool cellular components filters were then mounted and sealed into the end of a piece of PVC tubing. The cigarettes were lit, and a vacuum pump was then used to draw smoke backwards through the cigarette filters as the tobacco burned.

Before exposure to drawn cigarette smoke, the cellulose filter and wool cellular components were both white. During the burning of the cigarettes, the cigarettes with the wool cellular components filter burned much slower compared to those with the cellulose filter. After exposure, the cellulose filter was observed to be yellow/brown along the length of the filter. The wool cellular components filter material was yellow/brown on the side closest to the tobacco, but the opposite end was still white. This observation suggests that the wool cellular components are better at capturing particulates and tar from drawn cigarette smoke. Scanning electron microscopy (SEM) images showed that a thick coating is formed on the yellow/brown cellulose fibres and the yellow/brown wool cellular components after exposure to cigarette smoke.

### Example 6—Moisture Absorption by Wool and Wool Cellular Components Before and after Treatment with Ozone

Dry wool and wool cellular components were treated with ozone for 60 minutes. Wet wool cellular components were treated with ozone for 180 minutes. The ozone was generated using a room deodoriser and the ozone concentration in the flow was approximately 160-180 ppm.

The ability of all the materials to absorb 0.9% saline solution was measured against a pressure of 0.5 psi (3.45 kPa) using a standard Absorption Under Load (AUL) protocol. A glass cylinder was used with a piece of nylon screen mesh of 57 μm pore size secured over the end with a cable tie. The wool or wool cellular components material (total mass 0.3 g) were poured into the cylinder and a weight (total mass 235 g) was slid inside the cylinder. This apparatus was weighed, then placed on top of a petri dish containing a 40 mm porosity 0 sintered glass disc with filter paper (grade 541, 22 μm pore size) cut to size on top. Saline solution at 0.9% was poured into the petri dish up to the top of the glass disc. The whole system was covered with a glass jar and left to soak for 60 minutes. After 60 minutes the apparatus was weighed again.

The Absorbency Under Load (AUL) (g/g) for each untreated or ozone treated wool or wool cellular components material was calculated by the difference in mass of the apparatus before and after soaking, divided by the mass of the wool or wool cellular components material added to the cylinder (see table below). Ozone treatment improved the ability of the wool cellular components to absorb saline solution.

TABLE 1
AUL of wool and wool cellular components
with and without ozone treatment.

| Material | Treatment | AUL (g/g) |
|---|---|---|
| Wool | None | 1.55 |
| Wool | Dry ozone (60 mins) | 1.27 |
| Wool cellular components | None | 5.87 |
| Wool cellular components | Dry ozone (60 mins) | 7.23 |
| Wool cellular components | Dry ozone (180 mins) | 6.71 |
| Wool cellular components | Wet ozone (180 mins) | 7.63 |

### Example 7—Use of Wool Cellular Components as a Foam Additive

Wool cellular components were added to rigid and flexible forms of polyurethane and isocyanate foams. The wool cellular components are added during foam formation to produce a foam enriched with wool cellular components.

In one experiment, 90 g of Part A of a commercially supplied flexible polyurethane two pot mixture was mixed with 90 g Part B of the same commercially supplied flexible polyurethane two pot mixture. Immediately after mixing the two parts, 10 g of wool cellular components were added and mixed in. The expansion of the foam continued over 5 minutes and the foam was cured overnight.

### Example 8—Absorption/Filtration of Nitrogen Dioxide (NO₂) Gas by Wool Cellular Components as a Foam Additive

The NO₂ absorption of flexible polyurethane foams containing 0 and 5% wool cellular components (w/w) was measured using a glass chamber (3.5 L volume) containing a 2 g sample of flexible polyurethane foam inside a metal wire mesh cage and a portable air quality monitoring device with an interchangeable NO₂ sensor. The NO₂ gas absorption capacity of the control system (with no flexible polyurethane foam in the metal wire mesh cage) was also tested for comparison.

NO₂ flowed into the chamber (with or without flexible polyurethane foam containing 0 or 5% w/w wool cellular components) from a commercially supplied gas cylinder (5 ppm NO₂) for 1.5 minutes. The chamber was then closed and the decrease in gas concentration inside the sealed chamber was subsequently monitored. In the absence of flexible polyurethane foam, the concentration of NO₂ detected in the chamber at 1.5 minutes was 0.90-1.28 ppm (nominal maximum detection limit of the NO₂ sensor was 1 ppm NO₂). After the chamber was closed, a small increase in NO₂ concentration was detected (maximum concentration 1.60-1.77 ppm at 2.5-3 minutes) before the gas concentration decayed to 1.01-1.06 ppm at 25 minutes, demonstrating that the control system (without flexible polyurethane foam) absorbed some NO₂.

A greater amount of NO₂ was absorbed from the system in the presence of flexible polyurethane foam. With flexible polyurethane foam (0% wool cellular components) in the chamber, the NO₂ concentration detected at 1.5 minutes (before the chamber was closed) was 0.94-1.15 ppm. After the chamber was closed, the NO₂ concentration increased to 1.53-1.59 ppm at 2.5 minutes before decaying to 0.24-0.28 ppm at 25 minutes. Accordingly, the flexible polyurethane foam with no wool cellular components absorbed significantly more NO₂ than the control system.

The addition of 5% wool cellular components (w/w) to the flexible polyurethane foam increased the amount of NO₂ gas absorbed. With flexible polyurethane foam containing 5% wool cellular components (w/w) in the chamber, the maximum NO₂ concentration measured before the chamber was closed was 0.95-1.05 ppm at 1.5 minutes. After the chamber was closed, a small increase in NO₂ concentration was detected (1.53-1.63 ppm at 2.5 minutes) decaying to 0.19-0.22 ppm at 25 minutes.

### Example 9—Removal of Metal Ions with Wool Cellular Components

Wool cellular components (1 g) were immersed in 50 mL of an aqueous solution containing 3 ppm CuSO₄. Copper test strips (Insta-TEST Strips, Cu 0-3 ppm) were used to quantify the amount of copper ions in the solution before and after immersion of the wool cellular components. After immersing the wool cellular components in the CuSO₄ solution for 2 minutes, the amount of copper detected in the solution decreased from 3 ppm to 0 ppm.

### Example 10—Use of Wool Cellular Components as a Paper Additive

Wool cellular components were used as an additive in an otherwise conventional paper made from wood pulp. The resulting paper retained the physical characteristics of conventional paper.

Wool cellular components and unbleached fibre-cement grade kraft wood pulp were diluted to 1.2% consistency in demineralised water and soaked overnight. The resulting slurries were then dispersed at 3000 rpm for 20 minutes, using a standard disintegrator as specified in TAPPI (Technical Association of the Pulp and Paper Industry) standard T205. The slurries were then further diluted to 0.3% consistency in demineralised water in plastic buckets. Consistencies were confirmed by filtration of slurries through Whatman 113 filters, on a 105° C. oven dried basis.

The slurries were then blended to achieve a ratio of 70% wool cellular components and 30% wood pulp by dry mass, based on the pre-determined consistencies, to yield 159 cm diameter handsheets on a Messmer sheet former at basis weights between 60 and 150 gsm, as per T205. The appropriate amounts of each slurry to yield single handsheets were pre-weighed into plastic jugs. A bonding agent (cationic starch (Q500, Manildra)) was prepared as a 1% w/v solution and dosed at between 0.5 and 10 mg/g on a dry handsheet solids basis. The required amount of bonding agent solution per handsheet was measured into a plastic jug and diluted 1:10 with sufficient deionised water. The jugs of slurry were then poured rapidly into the jugs of bonding agent to effect mixing; and this action was repeated for a total of 5 times. Finally, the jug contents were transferred to a handsheet maker and the sheets formed. Handsheets were couched (transferred to blotters), pressed, dried and conditioned as per T205 and TAPPI standard T402.

Conditioned handsheets were tested for moisture content, grammage, thickness (calliper), density/bulk, tensile strength, tearing strength, bursting strength and air permeance using TAPPI standards T550, T220, T494, T414, T 403 and T 460, respectively. While burst strength was lower for the 70% wool cellular component sheet compared to the 100% wood pulp sheet (0.75 compared to 1.36 kPa·m²/g), the air permeance of the 70% wool cellular component sheet was 5 times higher than that of the 100% wood pulp sheet (5 compared to 1 s/300 ml, 1.22 kPa). This indicated superior performance for the 70% wool cellular component sheet when used as a gas filtration medium for the removal of pollutant gases, utilizing the gas absorption properties as described above, as well as superior performance in metal ion removal when used in the filtration of liquids as described above.

The 70% wool cellular component sheet was found to be effective at absorbing oil vapour. At a flow rate of 18 L/min of an air stream comprising 15.3 L/min of clean air combined with 2.7 L/min of oil vapour aerosol passing through a 70% wool cellular component sheet with a surface area of 100 cm² and a face velocity of 0.03 m/s, a filtration efficiency of 94.8% was observed.

### Example 11—Use of Wool Cellular Components to Create a Network Structure

Wool cellular components were bound together using an adhesive, providing a highly porous, high bulk network structure that retained the absorbent characteristics of the original wool cellular components and had useful insulating properties.

Wool cellular components (2 g) were blended in an air stream with 20 ml of 5% cyanoacrylate adhesive in dichloromethane, presented as a fine spray using a glass spray nozzle at 20 psi. This arrangement provided a disrupted air stream of freely moving loose wool cellular components that interacted with the small solvent droplets, binding the wool cellular components together into a network. Microscopic examination showed the network consisted of wool cellular components bound together by the cyanoacrylate adhesive.

The bulk of the network was 5 times greater than that of the wool cellular components; that is a fixed mass of the network had a volume 5 times greater than that of the same mass of wool cellular components. The network was found to retain the gas absorption characteristics observed for the wool cellular components as described above. Accordingly, the physical form of the network is useful for preparing filter components for gas or liquid contaminant removal.

### Example 12—Use of Wool Cellular Component Network for Insulation Applications

A sample of wool cellular component network was evaluated for insulation performance compared to standard insulation products, including goose down and polyester fill, and was found to be effective as an insulation material.

The insulation properties were assessed by using a modified version of ASTM D1518: Thermal Resistance of Batting Systems using a hot plate. In still air conditions the insulation material sample was placed in a 150 mm area on a hot plate set to 35° C. under a hood. The material was heated for 60 minutes and then left to cool. The temperature of the material, the air temperature in the hood and the hotplate temperature were measured.

Table 2 shows the temperature of each of the materials after heating for 60 minutes and the difference to air temperature after 60 minutes cooling.

TABLE 2
Insulation performance of wool cellular component network.

| | Material temperature | Difference to air |
|---|---|---|
| Material | reached (° C.) | temperature (° C.) |
| Polyester | 38.13 | +2.50 |
| Down | 42.23 | +4.21 |
| Wool cellular component | 40.71 | +3.68 |
| network | | |

It is not the intention to limit the scope of the invention to the abovementioned examples only. As would be appreciated by a skilled person in the art, many variations are possible without departing from the scope of the invention as set out in the accompanying claims. Accordingly, those persons skilled in the art will understand that the above description is provided by way of illustration only and that the invention is not limited thereto.

1-10. (canceled)

11. The material as claims in claim 63, wherein the material is an absorbent product.

12. The material as claimed in claim 11, wherein the keratin fibre cellular components comprise oxidised keratin fibre cellular components.

13. The material as claimed in claim 11, wherein the product is a liquid absorbent product.

14. The material as claimed in claim 11, wherein the product is a personal hygiene product.

15. The material as claimed in claim 11, wherein the product is a medical product.

16. The material as claimed in claim 11, wherein the product is for absorbing blood.

17. The material as claimed in claim 11, wherein the product is for absorbing urine.

18. The material as claimed in claim 11, wherein the product is a gas absorbent product.

19-21. (canceled)

22. The material as claimed claim 18, wherein the gas is selected from SO₂, NO₂, CH₂O, or a mixture of any two or more thereof.

23. The material as claimed in claim 22, wherein the product is for passive absorption of gaseous pollutants.

24. The material as claimed in claim 63, wherein the material is a filter.

25. The filter as claimed in claim 24, wherein the keratin fibre cellular components comprise oxidised keratin fibre cellular components.

26. The material as claimed in claim 24, wherein the filter is a liquid filter.

27. The material as claimed in claim 24, wherein the filter is a gas filter.

28-57. (canceled)

58. The material as claimed in claim 63, wherein the material is a thermal insulation material.

59. The material as claimed in claim 58, wherein the keratin fibre cellular components comprise oxidised keratin fibre cellular components.

60-62. (canceled)

63. A material comprising keratin fibre cellular components, wherein the keratin fibre cellular components are keratin fibre cuticle cells, keratin fibre cortical cells, or a combination of keratin fibre cuticle and cortical cells; and wherein the material is a network structure, composite foam, or a paper.

64. The material as claimed in claim 63, wherein material is a network structure and the keratin fibre cellular components are bound with an adhesive.
